(19) United States
(12) Patent Application Publication
(10) Pub. No.: US 2024/0091299 A1
(43) Pub. Date: Mar. 21, 2024

(54) TOPICAL COMPOSITIONS FOR ENHANCING A MAN'S SEXUAL RESPONSIVENESS AND PERFORMANCE

(71) Applicant: The Hidden Vault LLC, Slidell, LA (US)

(72) Inventor: Temeka Jones, Slidell, LA (US)

(73) Assignee: The Hidden Vault LLC, Slidell, LA (US)

(21) Appl. No.: 17/947,012

(22) Filed: Sep. 16, 2022

(51) Int. Cl. A61K 36/9068; A61K 9/00; A61K 31/05; A61K 36/068; A61K 36/185; A61K 36/258; A61K 36/31; A61K 36/41; A61K 36/63; A61K 36/746; A61K 36/79; A61K 36/82; A61K 36/8962

(52) U.S. Cl. CPC A61K 36/9068; A61K 9/0034; A61K 31/05; A61K 36/068; A61K 36/185; A61K 36/258; A61K 36/31; A61K 36/41; A61K 36/63; A61K 36/746; A61K 36/79; A61K 36/82; A61K 36/8962

(57) ABSTRACT

The present invention provides compositions formulated for topical administration to a penis of a person to enhance sexual responsiveness in the person, to enhance sexual performance, erection and stamina, and to increase the physical size of the penis, where the compositions comprise active components from herbal or plant origin including maca, tongkat ali, cordyceps, ginseng, fructus schisandrae. morinda root, rhodiola, rosea cordyceps sinensis, ginger, jasmine, sandalwood, leeks, camellia seed, jojoba oil and cannabidiol. The preferred composition is in the form of a massage oil, to be applied topically to the skin of the penis.

### BACKGROUND OF THE INVENTION

### Field of the Invention

The present invention generally relates to topically-administered compositions for the enhancement of sexual responsiveness, and more particularly, relates to a topically-administered compositions for enhancing sexual arousal, increasing libido, promoting growth, and promoting blow flow in the capillary network of the penis and of the mucosa of the penis glans.

### Description of the Related Art

Penile erection results from a complex interplay between central nervous (cerebral and spinal) and local factors. During erection, the penis increases in volume by the accumulation of blood, and an angle between 0 and 45 from the horizontal plane is created in the upright position. In this state, the shaft of the penis feels rigid, and the intracavernous pressure is close to the mean arterial blood pressure. Blood vessels, intrinsic smooth muscles of the penis, and surrounding striated muscles are controlled by nerves arising from three different parts of the peripheral nervous system: the thoracolumbar sympathetic, the lumbosacral parasympathetic, and lumbosacral somatic. Normal erection requires participation of all these systems. However, defects of any of them in a man result in decreased sexual performance, and at times, result in an inability to successfully perform intercourse at all.

Thus, in the physiology of an erection, nerve impulses which signal certain muscles to relax. These muscles, which are usually contracted, restrict arteries in the penis. When relaxed, the muscles permit a significant increase in blood flow. s Three groups of erectile tissue within the penis become engorged with blood and, in turn, less flaccid. The filled tissue and the muscle structure of the penis depress adjacent veins. The flow of blood out of the penis is thus restricted which contributes to an erection. Damage to or weakening of these nerves and blood vessels is a physiological reason for impotency.

For instance, diabetes causes damage to both nerves and blood vessels. Thus, diabetes often causes impotency in patients. A significant percent of all diabetic men suffer from impotency. The most widely used solution for impotency involves implants which are, in essence, internal prostheses. There are two basic types of such prostheses. One type utilizes internal semi-rigid rods which can be bent up or down. The second type involves a surgically implanted pump which transfers fluid from an implanted fluid reservoir to an inflatable device in the penis. These methods of correcting impotency have major shortcomings. The semi-rigid type does not allow the patient to control rigidity. The pump type requires hours of surgery on the penis, scrotum and abdomen and requires several days of hospitalization. Both types require surgery and are expensive. Also, once a prosthesis is implanted it makes a natural erection all but impossible.

External devices also have been used to produce and enhance an erection for men. Typically, these devices are tourniquet-like and fit tightly around the shaft of the penis. The flow of blood from the penis through the surface veins is thereby restricted. However, because the deep dorsal vein is generally not effectively depressed, blood exiting the penis is not satisfactorily restricted. Thus, the device's duration of effectiveness is relatively short. These external devices exhibit various other shortcomings including discomfort to both the user and sex partner and lack of efficacy in that the user might not achieve the desired usefulness as frequently as desired and to the extent preferred.

There are a wide variety of pharmacological agents used for the enhancement of erection and treatment of sexual dysfunction and as pro-libido agents. Some examples include: Serotonin receptor agonists and dopamine receptor agonists, adrenergic receptor agonists, inhibitors of phosphodiesterase; histamine receptor; neuropeptide antagonists; angiotensin II receptor antagonists; cholinesterase inhibitors; combinations of agents with the different types of biological activity; derivatives of vasoactive intestinal peptide; prostaglandins; antidepressants and antipsychotics; nitric oxide; calcitonin gene related peptide; and androgens.

Several compositions have been proposed in the past for treatment of male impotence or erectile dysfunction to promote sexual responsiveness. For example, U.S. Patent Application Publication No. 2007/0202199A1, filed by Barmensen Labs on Aug. 30, 2007, relates to a topical composition which enhances sexual responsive. Such topical composition comprises at least one active ingredient including methyl nicotinate, yohimbe, L-arginine in a pharmaceutically-acceptable topical vehicle. Also, U.S. Pat. No. 5,256,652 B2, issued to Pharmedic Co. on Oct. 26, 1993, provides a topical composition for enhancing the maintenance of penis erection comprising a peripheral vasodilator, a pharmacologically acceptable enhancer to facilitate absorption of the vasodilator, and a pharmacologically acceptable topical vehicle for the vasodilator. U.S. Pat. No. 6,500,440 B2, issued to Whan In Pharma Co. on Dec. 31, 2002, discloses a composition of topical preparation containing alprostadil, which has excellent skin permeation rate with little skin irritation, which is prepared by dissolving alprostadil or a Solid dispersion thereof prepared using poloxamer, into a mixture of an oily vehicle, a pyrrollidone and an anti-irritant agent.

While these conventional compositions may be suitable for the particular purpose employed, they would not be as suitable for the purposes of the present invention as disclosed hereafter.

Unfortunately, in addition, many or all of these pharmacological agents are associated with adverse effects including aggravation or induction of Schizophrenia, serotonin syndrome, central nervous system and endocrine system dysfunction, pain, echytomosis and priapism.

In humans, the penis is fully developed around the age of 21. While it is true that people can consider penis size to be a genetic trait, many other factors impact penis size. A case with a length of 4 cm or less at adulthood due to birth reasons or problems in the development process is generally called dwarf penis. It can lead to a lack of self-confidence and serious problems, such as suffering from interpersonal avoidance in social life. In other words, in men, the size of the penis is not limited to the problem of simply improving the satisfaction in the sexual aspect, and it occupies a very important part in terms of increasing self-confidence in interpersonal relationships and resolving emotional dissatisfaction.

Meanwhile, penis enlargement refers to various methods for increasing the size of a human male penis, and the goal of the method is to increase the length, thickness and circumference of the penis, and the size of the glans.

Methods for enlarging the circumference and length of the penis include a surgical method or a non-surgical method. Surgical methods include autologous dermal transplantation, autologous fat transplantation, replacement dermal transplantation, filler surgery, implant implantation, suspension ligament amputation, and the like, and non-surgical methods include using a decompression pump, a gelk exercise, and a traction device. Conventional over-the-counter creams, as well as supplements, vitamins, or mineral pills, that claim to increase penis size, simply do not achieve the intended result, and they often result in damage to the skin in the form of a rashe. Some of such conventional compositions also contain contaminants like pesticides, which may pose health risks.

Accordingly, there is a need for a penis enlargement formulation capable of effectively exhibiting a penile enlargement effect and effectively enhancing sexual endurance for men, as safe compositions, without causing side effects such as damage to the skin. There is also a need to provide a solution to enhance sexual responsiveness and arousal in the men.

Embodiments of the present invention disclosed herein provide for compositions and methods as disclosed herein and as defined in the annexed claims which provide for improved sexual responsiveness and performance by men, while also effectively exhibiting a penile enlargement and effectively enhancing sexual endurance by men.

### SUMMARY OF THE INVENTION

It is one prospect of the present invention to provide one or more novel compositions and methods of simple but effective formulation which can be topically applied to efficiently and effectively improve or enhance a man's sexual responsiveness as well as enlarge the size of the penis.

The following presents a simplified summary of the present disclosure in a simplified form as a prelude to the more detailed description that is presented herein.

Therefore, in accordance with embodiments of the invention, there is provided a composition formulated for topical administration to a penis of a human to enhance sexual responsiveness in the human comprising maca.

In one embodiment, the composition formulated for topical administration to a penis of a human to enhance sexual responsiveness in the human include active ingredients from herbal or plant origin, including maca, tongkat ali, cordyceps, ginseng, fructus schisandra, morinda root, rhodiola, rosea cordyceps sinensis, ginger, jasmine, sandalwood, leeks, camellia seed, jojoba oil and cannabidiol.

In one embodiment, the composition formulated for topical administration to a penis of a man to enhance sexual responsiveness in the man consists of 20% by weight of maca; 18% by weight of tongkat ali; 15% by weight of cordyceps; 15% by weight of ginseng; 10% by weight of fructus schisandrae; 10% by weight of morinda root; 5% by weight of rhodiola; 5% by weight of rosea cordyceps sinensis; and 2% by weight of ginger.

In a preferred embodiment, a composition formulated for topical administration to a penis of a man to enhance sexual responsiveness in the man is provided, consisting of 50% by weight of maca; 20% by weight of jasmine; 12% by weight of camellia seed; 8% by weight of sandalwood; 8% by weight of jojoba oil; and 2% by weight of leeks.

In another embodiment, the composition formulated for topical administration to a penis of a human to enhance sexual responsiveness in the human consists of 30% by weight of maca; 20% by weight of jasmine; 20% by weight of sandalwood; 10% by weight of leeks; 10% by weight of camellia seed; 5% by weight of jojoba oil; and 5% by weight of cannabidiol (CBD).

In a preferred embodiment, the composition of the invention is a massage oil. The composition formulated for topical administration to a penis of a man to enhance sexual responsiveness in the man includes active ingredients of herbal or plant origin to heighten the romantic experiences and to extend the period of intimacy.

In yet another embodiment, the invention provides a method for causing a penis to expand to erection by applying the composition of the present invention topically onto the skin of a penis, whereby the topical composition increases the sphygmic activity and blood stream of the arteries of the penis and causes the penis to swell to erection.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which like numerals throughout the FIGURES identify substantially similar components, in which:

FIG. 1A is a partially cross-sectional perspective view of the human penis before a massage of the penis with the compositions of the present invention, in accordance with embodiments of the invention; and

FIG. 1B is a partially cross-sectional perspective view of the human penis in erected position, after a massage of the penis with the compositions of the present invention, in accordance with embodiments of the invention.

### DETAILED DESCRIPTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein, the terms “formulation” and “composition” are used interchangeably and refer to a mixture of two or more compounds, elements, or molecules. In some aspects, the terms “formulation” and “composition” may be used to refer to a mixture of one or more active ingredients with a carrier or other excipients. The compositions of the present invention are used for topical administration specifically to penis of a male human and can be in the form of a solution, suspension, lotion, ointment, massage oil or any other composition used to be applied topically. Preferably, the composition is in the form of a massage oil applied as topical administration to a human penis.

The terms “extract of active ingredients” or “extract” used herein are used interchangeably. The extract is obtained from active ingredient from plant origin or herbal origin. The extract is prepared by extracting plant origin active ingredient or herbal material with suitable liquid. Typically, the extraction liquid is water or an alcohol.

The present invention provides a composition formulated for topical administration to a penis of a male human to enhance sexual responsiveness in the human includes extracts or active ingredients from plant origin. In one embodiment, the composition for topical administration according to present invention contains extracts of the active ingredients. Preferably, the extracts are prepared by collecting high quality raw materials and preparing the extract by vacuum distillation. The vacuum distillation replaces solvent extraction.

The active ingredients used in the composition for topical administration are preferably 100% organic plant-based, and due to this, they are free from side effects associated with conventional pharmacological agents used to increase the sexual responsiveness in men. The active ingredients which are used in the topical formulations of embodiments of the invention include one or more of the following: maca, tongkat ali, cordyceps, ginseng, fructus schisandrae, morinda root, rhodiola, rosea cordyceps sinensis, ginger, jasmine, sandalwood, leeks, camellia seed, jojoba oil, cannabidiol (CBD) and are described below. Herbal extracts containing each of the components that are suitable for use in formulating compositions of the present invention are available commercially.

Maca is a plant that grows in central Peru in the high plateaus of the Andes Mountains, and maca is an edible herbaceous biennial plant of the family Brassicaceae that is native to South America. It has been cultivated as a vegetable crop in that area for at least 2000 years. Maca is a relative of the radish and has an odor similar to butterscotch. Maca is also called Ginseng Andin, Peruvian Ginseng, Lepidium meyenii, or Lepidium peruvianum. Maca is a staple food of Peru that has also traditionally been used for its purported ability to regulate physical, mental, and emotional stresses in the body. People also take maca by mouth for female hormone imbalance, menstrual problems, symptoms of menopause, weak bones (osteoporosis), depression, stomach cancer, leukemia, HIV/AIDS, tuberculosis, and to boost the immune system. Maca root, the part of the plant most commonly used, contains fiber, amino acids, vitamins, and minerals. Through embodiments disclosed herein, the inventor has found that, as included in the compositions disclosed herein, maca enhances sexual arousal and performance, increases libido, as well as increases the size of a man's penis, as disclosed in the compositions herein, particularly when the composition is applied topically to a man's penis as a massage oil. Preferably, the composition of the present invention consists of 20% by weight of maca. In a preferred embodiment, as a massage oil, 5 drops of the disclosed composition is applied to the skin of the penis twice daily using a conventional dropper, and the massage oil is massaged into the skin for approximately 5 to 10 minutes for each application, in accordance with preferred embodiments of the invention.

Eurycoma longifolia, or tongkat ali, is a shrubby tree native to Southeast Asia. The plant is indigenous to Malaysia, Indonesia, and Vietnam. Some species of the plant also grow in regions of Cambodia, Myanmar, and Thailand. E. longifolia. It is used to relieve the following conditions as malaria, anxiety, intestinal worms, diarrhea, aging, itching, dysentery, constipation, exercise recovery, fever, diabetes, cancer, jaundice, lumbago, indigestion, leukemia, aches and pains, syphilis, osteoporosis. In one embodiment, the composition of the invention consists of 18% by weight of tongkat ali. Herbal extracts containing tongkat ali that are suitable for use in formulating compositions of the present invention are available commercially.

Cordyceps is a fungus that lives on certain caterpillars in the high mountain regions of China. Most cordyceps supplements are made in a lab. As disclosed herein, the inventor has found that, as included in the compositions disclosed herein, cordyceps helps improve immunity by stimulating cells and specific chemicals in the immune system. It might also help fight cancer cells and shrink tumor size, particularly with lung or skin cancers. Natural cordyceps is difficult to obtain at times and is expensive. People most commonly use cordyceps for athletic performance, kidney disorders, and liver problems, but there is no good scientific evidence to support these uses. In one embodiment, the composition of the invention consists of 15% by weight of cordyceps. Through embodiments disclosed herein, the inventor has found that, as included in the compositions disclosed herein, cordyceps helps to increases the size of a man's penis as well as enhance sexual arousal and performance, as disclosed in the compositions herein, particularly when the composition is applied topically to a man's penis as a massage oil. Herbal extracts containing cordyceps that are suitable for use in formulating compositions of the present invention are available commercially.

Ginseng refers to 11 different varieties of a short, slow-growing plant with fleshy roots. Ginseng may restore and enhance well-being. The herb has a light-colored, forked-shaped root, a relatively long stalk, and green leaves in an oval shape. Both American ginseng (Panax quinquefolius, L.) and Asian ginseng (P. Ginseng) may boost energy, lower blood sugar and cholesterol levels, reduce stress, promote relaxation, and help treat diabetes in humans. In one embodiment, the composition of the invention consists of 15% by weight of ginseng. Herbal extracts containing ginseng that are suitable for use in formulating compositions of the present invention are available commercially.

Schisandra (Schisandra chinensis)(or fructus schisandrae) is a plant native to northern China. The fruit is eaten as food and also used to make medicine. Schisandra is considered an adaptogen. Adaptogens are a class of natural substances that are believed to stimulate the body's resistance to physical, environmental, and emotional stressors. As disclosed herein, the inventor has found that, as included in the compositions disclosed herein, the chemicals in schisandra also improve liver function and increase energy, which improves overall endurance and coordination of a human. In one embodiment, the composition of the invention consists of 10% by weight of fructus schisandrae.

Morinda officinalis How and its root (“morinda root”) have long been used in traditional Chinese medicines in China and northeast Asia as tonics for nourishing the kidney as well as strengthening yang, the active male principle of the universe, characterized as male and creative and associated with heaven, heat, and light. It has also been conventionally used in Chinese medicine for strengthening bones and enhancing immunofunction in the treatment of osteoporosis, depression and inflammatory diseases such as rheumatoid arthritis and dermatitis. As disclosed herein, the inventor has found that, as included in the compositions disclosed herein, Morinda officinalis How and its root enhances sexual arousal and performance as well as contributes to increases in the size of a man's penis, through embodiments of the invention, particularly when the disclosed composition is applied topically to a man's penis as a massage oil.

Rhodiola (Rhodiola rosea) is a plant that grows in cold parts of Europe and Asia. Rhodiola is considered an adaptogen. As disclosed herein, the inventor as found that Rhodiola extract helps protect cells from damage and helps to regulate heartbeat. People use rhodiola for fatigue, anxiety, depression, stress, and many other conditions. In one embodiment, the composition of the invention consists of 5% by weight of rhodiola.

Ginger root comes from the Zingiber officinale plant, and it has been used in Chinese and Indian medicine for thousands of years. Ginger may help relieve nausea and vomiting and aid digestion. Antioxidants and other nutrients in ginger root may help prevent or treat arthritis, inflammation, and various types of infection. Ginger may also reduce the risk of diabetes, cancer, and other health problem. In one embodiment, the composition of the invention consists of 2% by weight of ginger.

Jasmine is a plant. The jasmine flower is used to make medicine. Jasmine has been used for liver disease (hepatitis), pain due to liver scarring (cirrhosis), and abdominal pain due to severe diarrhea (dysentery). It is also used to prevent stroke, to cause relaxation (as a sedative), and to aid in cancer treatment. Jasmine is often inhaled to improve mood, reduce stress, and reduce food cravings. Preferably, the composition of the present invention consists of 20% by weight of jasmine. Through embodiments disclosed herein, the inventor has found that, as included in the compositions disclosed herein, jasmine contributes to enhancing sexual arousal and performance and increases libido, as disclosed in the compositions herein, particularly when the composition is applied topically to a man's penis as a massage oil.

Sandalwood refers to the wood (heartwood) from tree species belonging to the family Santalaceae and genus Santalum. The wood from these trees has a characteristic aromatic fragrance. There are many species of trees that fall under this genus, of which the East Indian sandalwood (Santalum album) and Australian sandalwood (S. spicatum) are the most prominent. Traditionally, it is known for its use in perfumes, rituals, and a few medical conditions. It is used in medicine due to its antioxidant, anti-proliferative, and anti-microbial properties. The CAS No. of sandalwood is 84787-70-2. Preferably, the composition of the present invention consists of 8% by weight of sandalwood. Through embodiments disclosed herein, the inventor has found that, as included in the compositions disclosed herein, sandalwood contributes to enhancing sexual arousal, as disclosed in the compositions herein, particularly when the composition is applied topically to a man's penis as a massage oil.

Leeks look like giant green onions and can stand in for onions in many dishes. Historically, the early Greeks and Romans were fond of leeks, thinking they were good for the voice. Leeks are rich in flavonoids, especially one called kaempferol. Flavonoids are antioxidants and may have anti-diabetic, and anticancer properties, as well as other health benefits. The CAS No. of Leeks is 84650-15-7. Preferably, the composition of the present invention consists of 2% by weight of leeks. Through embodiments disclosed herein, the inventor has found that, as included in the compositions disclosed herein, leeks contributes to enhancing sexual arousal, as disclosed in the compositions herein, particularly when the composition is applied topically to a man's penis as a massage oil.

Camellia is a genus of flowering plants in the family Theaceae. They are found in eastern and southern Asia, from the Himalayas east to Japan and Indonesia Camellia Seed Carrier Oil is a highly nourishing, antioxidant-rich oil derived from the evergreen Camellia oleifera botanical native to the eastern regions of Asia. Preferably, the composition of the present invention consists of 12% by weight of camellia seed. In a preferred embodiment, as a massage oil, 5 drops of the disclosed composition is applied to the skin of the penis twice daily, by massaging the oil into the skin for approximately 5 to 10 minutes for each application, in accordance with the preferred embodiment of the invention.

Jojoba is a shrub that is grows in dry regions of northern Mexico and the southwestern United States. Jojoba oil and wax are produced from the seeds and used for medicine. Jojoba is applied directly to the skin for acne, psoriasis, sunburn, and chapped skin. It is also used topically to encourage the regrowth of hair in people who are balding. The CAS No. of Jojoba oil is 61789-91-1. Preferably, the composition of the present invention consists of 8% by weight of camellia seed. Through embodiments disclosed herein, the inventor has found that, as included in the compositions disclosed herein, Jojoba promotes sexual arousal and performance and increases libido, as disclosed in the compositions herein, particularly when the composition is applied topically to a man's penis as a massage oil.

Cannabidiol (CBD) is a chemical in the Cannabis sativa plant, also known as cannabis or hemp. As disclosed herein, the inventor has found that CBD enhances sexual arousal and performance and stamina, as disclosed in the compositions herein, particularly when applied topically to a man's penis as a massage oil, as the CBD works overtime to boost blood flow which extends the performance. Preferably, CBD infusion is used in embodiments of the invention. In one embodiment, the composition of the present invention consists of 5% by weight of CBD.

The inventors of the present invention have found that when specific percentages of above active ingredients or extracts mentioned above are mixed to prepare compositions for topical administration to a penis of a human, then there is enhancement in the sexual responsiveness. The compositions heighten romantic experiences, enlarge the size of the penis, and extend the lifetime of intimacy. The composition of the present invention are also used as an anti-inflammatory, which improves prostate health and improves stamina. The compositions also increase the volume and speed of blood flow in the capillary network of the cells of the penis, of the skin covering the penis, and of the mucosa of the penis glans, and they are able to promote growth enhancement.

The composition of the present invention enhances the sexual responsiveness in men by following three ways.
The first branch: The short penis is mainly due to damage to the corpus cavernosum cells, inhibition of cell division, so the first branch generally feels little effect, as the compositions mainly repair the corpus cavernosum tissue.
The second branch: Blood circulation to speed up the erection speed, to enhance the hardness of the blood vessels, to improve the thickening of the penis, and mainly to promote the sponge cell division, which leads to penile growth of 1-2 cm thickening; and the compositions increase the duration of sexual intercourse.
The third branch: To consolidate the expansion of the corpus, to strengthen metabolism, to enhance and increase the length of the penis length by 3-5 cm and increase the thickness of the penis by approximately 0.5-1 cm.

The concentration of each active ingredient or extract in the composition for topical administration is up to about 50%, preferably the concentration of each active ingredient or extract in the composition ranges up to about 40%, most preferably up to about 30%. The composition is made up of extracts from natural herbs or plant extracts.

In one embodiment of the invention, a composition formulated for topical administration to a penis of a human to enhance sexual responsiveness in the human includes 20% by weight of maca; 18% by weight of tongkat ali; 15% by weight of cordyceps; 15% by weight of ginseng; 10% by weight of fructus schisandrae; 10% by weight of morinda root; 5% by weight of rhodiola; 5% by weight of rosea cordyceps sinensis; and 2% by weight of ginger. In one embodiment, the composition is produced in a capsule form.

In a preferred embodiment, a composition formulated for topical administration to a penis of a human to enhance sexual responsiveness in the human includes 50% by weight of maca; 20% by weight of jasmine; 12% by weight of camellia seed; 8% by weight of sandalwood; 8% by weight of jojoba oil; and 2% by weight of leeks.

In another embodiment of the invention, a composition is formulated for topical administration to a penis to enhance sexual responsiveness in the human. The composition includes 30% by weight of maca; 20% by weight of jasmine; 20% by weight of sandalwood; 10% by weight of leeks; 10% by weight of camellia seed; 5% by weight of jojoba oil; and 5% by weight of cannabidiol (CBD). In an embodiment, the CBD is added from its original format.

In another embodiment of the invention, a method for causing a penis to expand to erection is disclosed. The method initiates with a step of applying a composition consisting of either topical composition (a) or topical composition (b), as follows.

The topical composition (a) includes: 20% by weight of maca, 18% by weight of tongkat li, 15% by weight of cordyceps, 15% by weight of ginseng, 10% by weight of fructus schisandrae, 10% by weight of morinda root, 5% by weight of rhodiola, 5% by weight of rosea cordyceps sinensis, and 2% by weight of ginger.

The topical composition (b) includes 30% by weight of maca, 20% by weight of jasmine, 20% by weight of sandalwood, 10% by weight of leeks, 10% by weight of camellia seed, 5% by weight of jojoba oil, and 5% by weight of cannabidiol (CBD).

The method includes a step of applying the topical composition (a) or (b) topically onto the skin of the penis, whereby said topical composition (a) or topical composition (b) increases the sphygmic activity and blood stream of the arteries of the penis and causes the penis to swell to erection. In a preferred embodiment, the topical composition (a) and the topical composition (b) is in the form of a massage oil.

In a preferred embodiment, the the step (b) of applying the topical composition (a) or (b) topically onto skin of the penis includes applying the topical (a) or (b) topically onto skin of the penis two times each day.

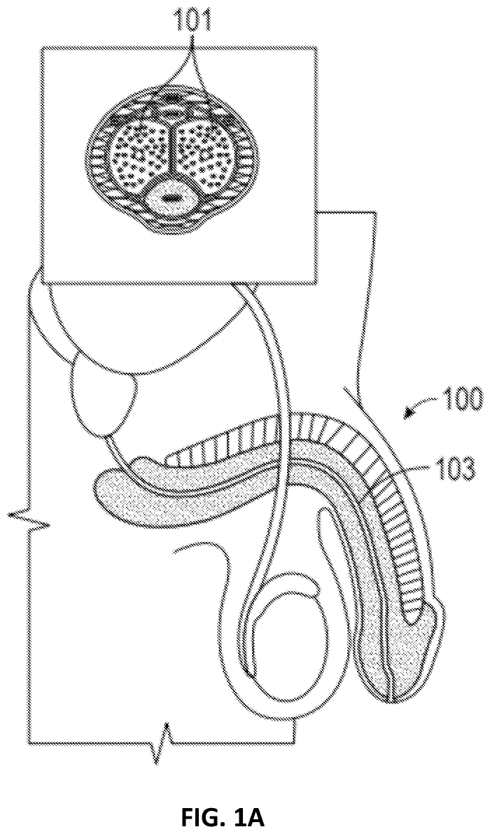

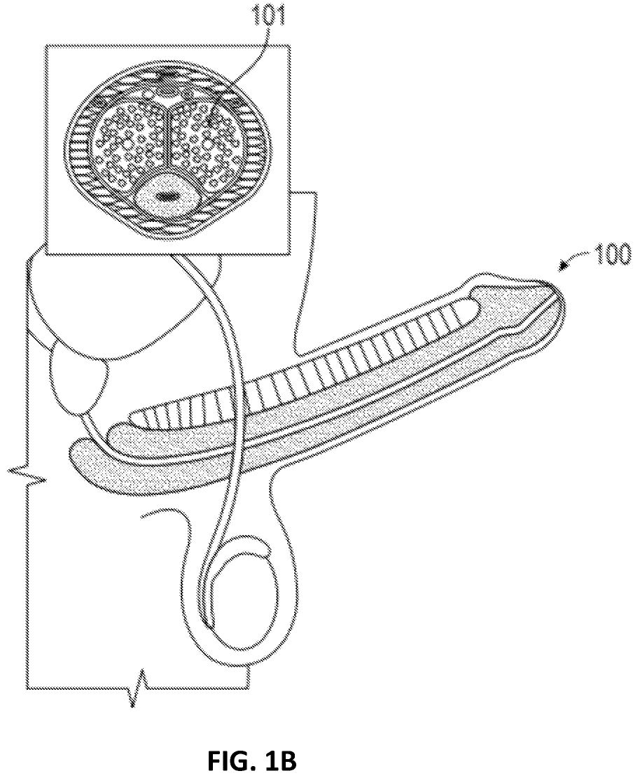

Referring initially to FIGS. 1A-1B, the basic impact of the compositions of the present invention on human penis, according to preferred embodiments of the present invention is discussed.

In FIG. 1A, a partially cross-sectional perspective view of the human penis 100 before massage with the composition of the present invention is shown, in accordance with embodiments of the invention. Human penis 100 generally includes of urethra 103 for the passage of urine and semen. A cross sectional view of human penis 100 shows a pair of corpora cavernosa 101, which are the spongy tissues in the human penis 100. Human penis 100 is small mainly due to the damage to the corpus cavernosum cells, or as a result of an expression of a person's particular genetics. The compositions of the invention repair the corpus cavernosum tissue, to promote growth of the penis. The compositions of the invention also enhance cell division and blood flow within the penis to promote growth of the penis.

FIG. 1B is a partially cross-sectional perspective view of the human penis 100 in erected position, after massage with the composition of the present invention, in accordance with embodiments of the invention. Preferably, a user applies 5 drops of the composition either in the form of oil or gel, and the user massages the composition onto the penis twice a day for preferably 5 to 10 minutes, or until the composition is completely absorbed into the skin of the penis. The results are visible and as exemplified in FIG. 1B, the human penis 100 is now enlarged both in length and thickness as a result of the application of the compositions disclosed herein.

Generally, the inventor has found that, within in a short span of time, in addition to helping a man's penis become erect, a topical application to a male's penis of the compositions disclosed herein, preferably two times each day, results in an increase in penile thickness between a range of 0.5 cm to 2 cm and results in an increase in the penile length between a range of 2 cm-5 cm, when compared to the previous size of the penis without any application of the compositions disclosed herein.

It is advisable to use protective gloves while massaging and to avoid eating, drinking and smoking while topically massaging the composition onto the penis. Further, the user should preferably wash their hands before eating, drinking and smoking in order to avoid direct contact of the composition with the user's mouth or nose.

The composition is preferably sold in a container. It is recommended to use the original container and to store the composition in a cool environment and in accordance with any local regulations. The container should be protected from direct sunlight in a dry, cool and well-ventilated area, away from incompatible materials and food and drink. The container is preferably stored in an environment having a temperature ranging from 5 to 40° C. (i.e., from 41° F. to 104° F.).

The preferred composition is in the form of a massage oil comprising a colorless liquid, with a mild odor. The composition has a 93° C. (i.e., 199.4° F.) flash point, and it is insoluble in water. The composition is chemically stable under recommended storage and handling conditions. The composition needs to be avoided from oxidizing agent materials, direct sunlight, high temperatures and open fire. The composition might be irritating to skin of certain allergic persons, and it may cause eye irritation in case of direct contact with a user's eyes.

The composition may cause respiratory sensitization when in direct contact. Further, the composition has not shown any germ cell mutagenicity, carcinogenicity properties, reproductive toxicity, organ toxicity, aspiration hazard, and Eco-toxicity. Further, the composition is naturally biodegradable, has low bio accumulation of the material in aquatic organisms, and it is dissolvable in soil material.

The present application offers various advantages such as a 100% plant extract composition for expanding the size of the penis and to enhance sexual responsiveness in a male human. The composition increases the length of penis between 2 cm-5 cm and increases the thickness of the penis by 0.5 cm-2 cm. The composition is natural (meaning, without man-made hazardous chemicals) and is free from side effects to the user. Through embodiments of the invention, the compositions of the present invention have a shelf life of thirty-six (36) months.

As can be appreciated from the disclosure herein, through preferred embodiments of the invention, the disclosed composition formulated for topical administration to a penis to enhance sexual responsiveness in a human not only treats erectile dysfunction by improving blood flow, the composition also stimulates arousal, promotes sexual performance, and increases the physical size of the penis when erect.

The drawings and the forgoing description give examples of embodiments. Those skilled in the art will appreciate that one or more of the described elements may be very well combined into a single functional element. Alternatively, certain elements may be split into multiple functional elements. Elements from one embodiment may be added to another embodiment. For example, the orders of the processes described herein may be changed and are not limited to the manner described herein.

Also, those acts that are not dependent on other acts may be performed in parallel with the other acts. The scope of embodiments is by no means limited by these specific examples. The components of the system including related technologies mentioned above are collectively used to improve performance of the composition.

Numerous variations, whether explicitly given in the specification or not, such as differences in formulation, fraction, and use of material, are possible. Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

All U.S. patents and publications identified herein are incorporated in their entirety by reference thereto.

The claimed invention is:

1. A composition formulated for topical administration to a penis to enhance sexual responsiveness in a human, consisting of:
20% by weight of maca;
18% by weight of tongkat ali;
15% by weight of cordyceps;
15% by weight of ginseng;
10% by weight of fructus schisandrae;
10% by weight of morinda root;
5% by weight of rhodiola;
5% by weight of rosea cordyceps sinensis; and
2% by weight of ginger.

2. A composition formulated for topical administration to a penis to enhance sexual responsiveness in a human, consisting of:
50% by weight of maca;
20% by weight of jasmine;
12% by weight of camellia seed;
8% by weight of sandalwood;
8% by weight of jojoba oil; and
2% by weight of leeks.

3. A composition formulated for topical administration to a penis to enhance sexual responsiveness in a human, consisting of:
30% by weight of maca;
20% by weight of jasmine;
20% by weight of sandalwood;
10% by weight of leeks;
10% by weight of camellia seed;
5% by weight of jojoba oil; and
5% by weight of cannabidiol (CBD).

4. A method for causing a penis to expand to erection, the method comprising:
(a) providing a topical composition consisting of either topical composition (a) or topical composition (b),
wherein topical composition (a) consists of:
50% by weight of maca;
20% by weight of jasmine;
12% by weight of camellia seed;
8% by weight of sandalwood;
8% by weight of jojoba oil; and
2% by weight of leeks;
wherein topical composition (b) consists of:
30% by weight of maca;
20% by weight of jasmine;
20% by weight of sandalwood;
10% by weight of leeks;
10% by weight of camellia seed;
5% by weight of jojoba oil; and
5% by weight of cannabidiol (CBD); and
(b) applying the topical composition (a) or (b) topically onto skin of the penis, whereby said topical composition (a) or topical composition (b), said application of topical composition (a) or (b) adapted to increase the sphygmic activity and blood stream of the arteries of the penis and cause the penis to swell to erection.

5. The method of claim 4, wherein the step (b) of applying the topical composition (a) or (b) topically onto skin of the penis comprises applying the topical (a) or (b) topically onto skin of the penis two times each day.

6. The method of claim 4, wherein the step (b) of applying the topical composition (a) or (b) topically onto skin of the penis comprises applying 5 drops of the topical (a) or (b) topically onto skin of the penis two times each day.

7. The method of claim 4, wherein the step (b) of applying the topical composition (a) or (b) topically onto skin of the penis comprises applying 5 drops of the topical (a) or (b) topically onto skin of the penis two times each day, wherein said application comprises massaging the topical composition (a) or (b) onto the skin of the penis twice a day for a period of 5 to 10 minutes.
